(19) United States
(12) Patent Application Publication
(10) Pub. No.: US 2009/0208464 A1
(43) Pub. Date: Aug. 20, 2009

(54) MESENCHYMAL STEM CELL ISOLATION AND TRANSPLANTATION METHOD AND SYSTEM TO BE USED IN A CLINICAL SETTING

(76) Inventor: Christopher J. Centeno, Westminster, CO (US)

Correspondence Address: SWANSON & BRATSCHUN, L.L.C., 8210 SOUTHPARK TERRACE, LITTLETON, CO, 80120, US

(21) Appl. No.: 12/161,911

(22) PCT Filed: Jan. 23, 2007
(86) PCT No.: PCT/US2007/060889

§ 371 Date: Nov. 7, 2008

Related U.S. Application Data
(60) Provisional application No. 60/761,441, filed on Jan. 24, 2006.

(51) Int. Cl. A61K 35/12

(52) U.S. Cl. 424/93.7

(57) ABSTRACT

A system and method for the percutaneous, autologous transplantation of mesenchymal stem cells and progenitor helper cells (PHC) from bone marrow to degenerated intervertebral discs or joints. This method is designed to be used by operating room staff in a clinical setting to isolate a mesenchymal stem cell population and PHC during the same surgical procedure as transplantation. The method can be used as a two step procedure where cells are harvested, then isolated, then reimplanted at a later time. In addition, experimental techniques are described to determine which bone marrow cells should be removed via negative selection to generate a PHC population most likely to regenerate certain tissue types in-vitro as well as which combination of fibrinogen and hyaluronic acid and which degree of gel maceration provides the best matrix for in-vitro and in-vivo regeneration of joints and intervertebral discs.

### RELATED APPLICATIONS

This application claims priority to U.S. Provisional Application Ser. No. 60/761,441, filed Jan. 24, 2006, entitled, “Mesenchymal Stem Cell Isolation And Transplantation Method And System To Be Used In A Clinical Setting,” which is incorporated by reference in its entirety.

### TECHNICAL FIELD

The present invention is directed toward a system and method for the transplantation of mesenchymal stem cells and in particular a system and method for the percutaneous, autologous transplantation of mesenchymal and progenitor helper cells from bone marrow to degenerated intervertebral discs or joints.

### BACKGROUND OF THE INVENTION

Mesenchymal stem cells (MSC's) have widely reported regenerative capabilities in animal models. (Acosta et al. (2005) Neurosurg Focus 19(3):E4; Barry (2003) Novartis Found Symp. 249: 86-102, 170-4, 239-41; Brisby et al. (2004) Orthop Clin North Am. 35(1): 85-93; Buckwalter and Mankin (1998) Instr Course Lect. 47: 487-504; Caplan (1991) J Orthop Res. 9(5): 641-50; Caplan and (2001) Trends Mol Med. 7(6): 259-64; Fortier et al. (1998) Am J Vet Res. 59(9): 1182-7; Gruber and Hanley (2003) Spine 28(2): 186-93; Johnstone and Yoo (1999) Clin Orthop Relat Res. 367 Suppl: S156-62; Luyten (2004) Curr Opin Rheumatol 16(5): 599-603; Magne et al. (2005) Trends Mol Med; Murphy et al. (2003) Arthritis Rheum. 48(12): 3464-74.) While these cells are now just entering clinical trials in humans, all methods described in the literature require specialized lab skills not found in hospital or clinic operating rooms. Similarly, no practical method of isolating these cells quickly by operating room staff has been developed. In addition, what has been reported as isolated MSC's in the literature usually not MSC's, but a heterogeneous population of nucleated cells of which only between 1 in 100 to 1 in 100,000 are actually MSC's.

Due to the size of many of the target structures to be regenerated (such as intervertebtral discs or articular facet joints of the spine), there is a practical need to enrich the MSC population for effective therapeutic use. Bone marrow stoma contains many different cell types including endothelial cells, platelets, red blood cells, monocytes, lymphocytes, macrophages as well as uncommitted progenitor cells of both hematopoetic and mesenchymal lineages. (Alhadlaq and Mao (2004) Stem Cells Dev. 13(4): 436-48.) Injecting nucleated cells obtained from a bone marrow source which do not participate in the regenerative process will dilute the absolute numbers of MSC's in any injectate. Since there have been no human clinical trials of intervertebral disc or joint regeneration published, the specific types of cells which should be injected to best allow repair of these structures is not known. For instance, it is not known if in-vivo human clinical trials will reveal that a certain density of MSC's is required, if other non-MSC cells are needed to support MSC's in the regenerative process, or if certain cells left in a nucleated cell isolate are deleterious to the regenerative process.

Certain references have suggested the use of MSC's in a laboratory setting to treat disease. No references have suggested a method that could be used by clinical providers without training and expertise in laboratory methods. While MSC based regenerative techniques hold great promise, physicians will be unlikely to utilize regenerative techniques unless the isolation can be easily performed by operating room staff and the isolation itself can be performed during the same surgical procedure as the actual transplantation. If expansion of the cells is required for success, then that expansion would preferably be carried out in a hospital or clinical lab and not a research laboratory.

Attawia (U.S. application 20040229786) describes an isolation technique for MSC's used to treat intervertebral disc disease. The Attawia method however, includes lysis of the RBC's and high grade centrifugation techniques which are not practical for operating room personnel. For instance, the types of RBC lysis discussed have very small margins of error. Thus Attawia does not teach or suggest a technique that can be used by operating room staff with wide margins of error. In addition, the Attawia methods only serve to isolate a heterogeneous population of nucleated cells and not MSC's. It has also been shown that there is a three fold decrease in osteogenic potential of MSC's obtained from middle aged and older patients when compared to patients under 36 years of age. (D'Ippolito et al. (1999) J Bone Miner Res. 14(7): 1115-22.) Since the population most in need of the regeneration of discs and joints is in fact middle aged or elderly, injecting a heterogeneous population of cells in these patients will only dilute the density and number of progenitor cells capable of tissue regeneration. The Attawia application makes no statement regarding which cell surface antigens or other properties of cells could be used to isolate MSC' s from the heterogeneous population of nucleated cells isolated using their techniques. In addition, it does not reveal the concept of isolating a “Progenitor Helper Cell” (PHC) population. While the Attawia application discusses the use of immunoabsorbtion, it does not detail which cell surface antigens are to be used to produce a cell population most likely to regenerate specific tissues.

Other methods previously described in U.S. Pat. No. 6,200,606 ('606 herein) require steps not practical for surgeons and hospitals such as in-vitro culture. This application also requires the use of a surgically implanted device. Negative selection techniques using cell surface antigens are discussed, but there is no discussion of which cells should be selected out using this technique to produce the desired tissue repair. In addition, the '606 patent discusses using complex laboratory techniques that would result in a heterogeneous population of nucleated cells being delivered back into a patient for tissue regeneration. In the patients in most need to tissue repair (the middle aged and elderly), this would result in massive dilution of cells capable of repairing tissue. If in-vitro culture expansion is required for success of this procedure, Peterson does not detail how such expansion could be carried out by a clinical or hospital lab without experienced research personnel.

In addition, immunoadsorption techniques remove cells from the heterogeneous marrow sample by exploiting the binding properties of monoclonal antibodies. The cell surface antigens of the cells to be selected preferentially bind to these antibodies which are attached to the surface of a bead, heavier chain of molecules, magnetic particle, or other device. Immunoadsorption techniques are popular in clinical applications and in research because they target cells with monoclonal antibodies and unlike fluorescence activated cell sorting (FACS), they can be scaled for the large numbers of cells in a clinical sample. In addition, Immunoadsorption techniques avoid the dangers of using cytotoxic reagents such as immunotoxins, and complement. There is also considerable cost and expertise needed to isolate cells using a FACS technique.

Currently available marrow cell progenitor isolation kits and devices are designed to select out CD34+ heme progenitors. Thomas et al have described a system (U.S. Pat. No. 6,872,567) whereby negative selection can be used to enrich MSC's in a simple fashion by using RBC's to form immunorosettes. MSC's are known not to express CD31, CD14, CD11a, CD45, glycophorin A, CD 3, CD 14, CD19, CD34, CD 38, CD66b. (Alhadlaq and Mao (2004) Stem Cells Dev. 13(4): 436-48) However, while immunoadsorption techniques can be used to select out these non-MSC cells, it is not known which cells should be selected out to produce the best clinical result. For example, Singer and colleagues have determined that both stromal and hematopoietic cells have common precursors. (Singer et al. (1987) Blood 70(2): 464-74; Singer et al. (1984) Leuk Res. 8(4): 535-45.) In addition, Huang and colleagues have seen that a fetal CD34+ cells can differentiate into both adult CD34+ hematopoietic cells and CD34− stromal cells. (Huang and Terstappen (1994) Nature 368(6472): 664.) Huss has also observed that fibroblast like CD34− cells can give rise to CD34+ cells with hematopoietic properties. In addition, the CD34 expression of early hematopoietic progenitors is reversible. (Sato et al. (1999) Blood 94(8): 2548-54.) In fact, Huss describes a dynamic stromal environment whereby quiescent stem cells may be activated by certain growth factors. (Huss (2000) J Hematother Stem Cell Res. 9(6): 783-93.) Other authors have observed that the presence of MSC's were needed for CD34+ cell expansion. This dictates that there is a complex epigenetic interaction between the two cell types. (Koh et al. (2005) Biochem Biophys Res Commun. 329(3): 1039-45

From a practical clinical perspective, it is unknown whether CD34+ cells should be selected out or left in a marrow sample that is intended to regenerate certain tissues. In addition, other cells such as platelets are known to contain naturally occurring growth factors such as PDGF-BB which impact MSC development. (Cashman et al. (1990) Blood 75(1): 96-101; Cassiede et al.(1996) J Bone Miner Res. 11(9): 1264-73; Fiedler et al. (2004) J Cell Biochem. 93(5): 990-8; Fiedler et al. (2002) J Cell Biochem. 87(3): 305-12; Katz et al. (1987) Leuk Res. 11(4): 339-44; Xaymardan et al. (2004) Circ Res. 94(5): E39-45; Zhu et al. (2005) Stem Cells. In addition, platelets have been shown to have varying effects on MSC's and other progenitor cells. (Cashman et al. (1990) Blood 75(1): 96-101; Cassiede et al.(1996) J Bone Miner Res. 11(9): 1264-73; Katz et al. (1987) Leuk Res. 11(4): 339-44; Kang et al.(2005) J Cell Biochem. 95(6): 1135-45; Miyata et al. (2005) J Cell Physiol. 204(3): 948-55; Kitoh et al. (2004) Bone 35(4): 892-8; Kilian et al. (2004) Eur J Med Res. 9(7): 337-44; Gruber et al. (2004) Platelets 15(1): 29-35; Hirschi et al. (1999) Circ Res. 84(3): 298-305; Reddi and Cunningham (1990) Biomaterials 11: 33-4.) Therefore, it is presently unknown whether platelets should be excluded or included in an enriched marrow sample where the MSCs are intended to proliferate in-vivo. It is presently unknown what other cells are PHCs, or which of these cells provide a helper role (have growth factors which can activate or differentiate MSCs). It is unknown which of these cells has a transforming role (can differentiate themselves to MSCs under the correct environmental conditions). It is unknown whether certain cells might be PHCs in certain relative concentration to MSCs and be helpful for tissue regeneration while being deleterious to MSC survival at other concentrations. Since the number of cells likely to be active progenitors in the elderly likely only represent 10-20% of 1 in 10,000 nucleated cells, knowing which cells function as PHCs in an autologous transplant and will thus promote the growth of the scarce MSC population (PHCs). In addition, this knowledge is critical to prevent massive dilution of the active agent (MSCs). Determination of which cells are in fact PHCs is unknown at this time, yet these findings may well play a role is the success of early human clinical trials where lab techniques such as TGF-beta stimulation with virus vectors (common in research settings) may not be practical or available for everyday human use for many years.

The use of autologous fibrin as a tissue engineering scaffold holds great promise. (Ruszymah (2004( Med J Malaysia 59 Suppl B: 30-1.) Synthetic fibrin glue has also been used as a scaffold material for mesenchymal stem cells in bony repair. (Oshima et al. (2004) Osteoarthritis Cartilage 12(10): 811-7; Fang et al. (2004) J Huazhong Univ Sci Technolog Med Sci. 24(3): 272-4; Yamada et al. (2003) J Craniomaxillofac Surg. 31(1): 27-33.) Silverman has determined that fibrin glue can be used as a three dimensional scaffold for chrondocytes in an animal model. (Silverman et al. (1999) Plast Reconstr Surg. 103(7): 1809-18.) Sah has investigated specific formulations of fibrinogen and thrombin on chrodrocytes and matrix formation. (Sah et al., Mankarious, EFFECTS OF FIBRIN GLUE COMPONENTS ON CHONDROCYTE GROWTH AND MATRIX FORMATION, in 49th Annual Meeting of the Orthopaedic Research Society.) Bens also investigated specific formulations for bone repair finding that 18 mg/ml of fibrinogen and a thrombin activity of 100 IU/ml was optimal for producing fibrin scaffolds that would allow appropriate MSC spreading and proliferation. (Bensaid et al. (2003) Biomaterials 24(14): 2497-502.) Park has tested a fibrin glue and hyaluronic acid (HA) composite with chrondrocytes in an animal model and found earlier cartilage formation, higher GAG, water, and collagen content with hyaluronic acid added. (Park et al. (2005) Artif Organs 29(10): 838-45.) Park used fibrinogen (9-18 mg/mL) and HA of molecular size 3000 kDa (10 mg/mL). The chondrocytes were then homogeneously mixed with aprotinin and 60 U/mL thrombin (1000 U/mg protein) and a fibrin stabilizing Factor XIII, as well as 50 mM CaCl2. No investigation was carried out with stem cells or with differing concentrations of HA, fibrin, and thrombin or different molecular weight HA's. At this time it is unknown if such a scaffold would work in a joint space or intervertebral disc with mesenchymal stem cells or if there is an optimum amount of thrombin, fibrinogen, and hyaluronic acid that would promote specific tissue repair. At this point, no research exists using this composite with mesenchymal stem cells.

Goldberg describes a process for the use of mesenchymal stem cells mixed with preferably a collagen matrix (but fibrin glue is also discussed) and delivered to a joint via an arthroscopic approach (U.S. Pat. No. 6,835,377). No discussion of a hyaluronic acid and fibrinogen composite is revealed. Whitmore has described the use of fibrin glue and hyaluronic acid for wound healing (U.S. Pat. No. 6,699,484), but does not entertain a fibrinogen and hyaluronic acid cell scaffold for the percutaneous delivery of stem cells. Radice (U.S. Pat. No. 6,699,471) has discussed the use of hyaluronic acid as a carrier for bioactive cells and a chondrocyte tissue repair scaffold, but not in combination with fibrinogen. U.S. Pat. Nos. 5,749,874 and 5,769,899 (both Schwartz et al 1998) disclose the use of biodegradable hyaluronic acid with a surgical implant. Mansmann (U.S. Pat. No. 6,530,956) discloses an anchoring device that is designed to hold a porous and flexible matrix, made of a material such as collagen or hyaluronic acid, which will hold chondrocyte cells. Again, this is a surgical implant augmented by hyaluronic acid and cells, but is not meant to be administered percutaneously. Naughton (U.S. Pat. No. 5,842,477) reveals the use of a hyaluronic acid and other material scaffold embedded with chondrocyte progenitors, but fibrinogen is not used as a composite with hyaluronic acid, but instead as an adhesive to hold the scaffold in place. Zheng has disclosed a broad patent application (# 20040136968) which does discuss the use of a biodegradable tissue scaffold with multiple autologous cells types. The use of MSC's among other cells is contemplated. This support matrix preferably includes collagen and or other materials including cells, hyaluronic acid, and possibly an unspecified fibrin Type. It does contemplate that cells and matrix could be delivered though an injection. It does discuss that cartilage repair in a joint is one goal of the invention. It does not reveal the use of this scaffold or these cells to repair an intervertebral disc. It does not disclose that fibrinogen will be mixed with hyaluronic acid to produce a composite outside of the body and then macerated before being mixed with cells and injected through a needle. Benette (U.S. patent application No. 20040078077) discloses the use of a biocompatible scaffold for multiple cell types including stem cells that may include fibrin and hyaluronic acid (separately but not in combination) for the potential regeneration of tendon and ligament injuries. In Binette (U.S. patent application No. 20050038520) fibrinogen and hyaluronic acid are mentioned among long list of possible combinations, not mentioned in specific combination as a sole composite, and this combination's use in Intervertebral disc regeneration is not discussed. Hill also reveals (U.S. patent application No. 20050118230) fibrinogen and hyaluronic acid among another long list of possible combinations for possible stem cell scaffold use. The use of this possible composite in the intervertebral disc not discussed. In none of these patents or applications is the use of a particular combination of fibrinogen, and hyaluronic acid discussed to produce a composite mesenchymal stem cell scaffold which is hardened outside the body and then macerated and injected percutaneously into the Intervertebral disc or a joint for cartilage repair.

The rationale for injecting substances via percutaneous needle placement is to reduce patient trauma. All prior applications discuss the use of self assembling gels or liquids which become gels once placed into the joint space or intervertebral disc. These gels would allow very little or rare opportunity for MSCs to migrate to contact existing nucleus pulposis or chondrocyte cells. For regenerating the intervertebral disc without the use of chemical agents such as TGF-beta this is important, as Richardson demonstrated that cell to cell contact is required to differentiate MSCs to a nucleus pulposis phenotype under these circumstances. (Richardson et al. (2005) Stem Cells.

The mechanics of pushing a hardened gel through a long 22 gauge needle can require significant force. As a result, for longer needle applications (such as the 22 gauge 6-9 inch needle required for fluoroscopically guided placement of cells into a lumbar intervertebral disc) a screw device on the top of the syringe is turned by the surgeon to push the gel through the long needle. However, such force can traumatize cells.

Discitis is a serious complication which can occur when cells are processed outside of the body and skin flora are seeded into the disc (Gibson and Waddell (2005) Spine 30(20): 2312-20), the use of antibiotics with percutaneous disc access procedures is usually advocated. However, Hoelscher has determined that antibiotics at higher concentrations have a negative impact on in-vitro disc cell metabolism. (Hoelscher et al. (2000) Spine 25(15): 1871-7.) However, the exposure to the air and other possible contaminants discussed in the laboratory focused techniques described by the Attawia application and Peterson patent produce a situation where antibiotics would be needed to prevent infection. In addition, Willems demonstrated that a two needle disc access technique did not produce significant infection even in the absence of antibiotics. (Willems et al. (2004) J Spinal Disord Tech. 17(3): 243-7.)

As already discussed, the Attawia application and '606 patent reveal the use of complex laboratory techniques that do not isolate MSC'S, but result in a heterogeneous population of nucleated cells. In doing so, the cells are exposed to the air and multiple containers including pipettes, centrifuge tubes, and other devices. In a laboratory setting a sterile hood would be used by laboratory and research staff to reduce the likelihood of bacterial contamination, however, such a hood does not exist in an operating room setting. Infectious discitis is a serious disease with very significant complications and treatment challenges. (Fujiwara et al. (1994) Neurol Med Chir (Tokyo) 34(6): 382-4; Iversen et al. (1992) Acta Orthop Scand. 63(3): 305-9; Ponte and McDonald (1992) J Fam Pract. 34(6): 767-71; Nielsen et al. (1990) Acta Radiol. 31(6): 559-63; Del Curling et al. (1990) Neurosurgery 27(2): 185-92; Borner and Follath (1989) Schweiz Med Wochenschr. 119(1): 19-21; Kambin and Schaffer (1989) Clin Orthop Relat Res 238: 24-34; Weber (1988) Z Orthop Ihre Grenzgeb 126(5): 555-62; Bircher, et al. (1988) Spine 13(1): 98-102; Dall et al. (1987) Clin Orthop Relat Res. 224: 138-46; Onofrio (1980) Clin Neurosurg. 27: 481-516.) In addition, many cases of peripheral and spinal joint septic arthritis have been reported due to percutaneous injection of various substances. (Charalambous et al. (2003) Clin Rheumatol. 22(6): 386-90; Chazerain et al. (1999) Rev Rhum Engl Ed. 66(7-9): 436; Gustafson et al. (1989) Am J Vet Res. 50(12): 2018-22; Kortelainen and Sarkioja (1990) Z Rechtsmed. 103(7): 547-54; Laiho and Kotilainen (2001) Joint Bone Spine 68(5): 443-4; Morshed et al. (2004) J Bone Joint Surg Am. 86-A(4): 823-6; Orpen and Birch (2003) J Spinal Disord Tech. 16(3): 285-7; Pellaton et al. (1981) Schweiz Rundsch Med Prax. 70(52): 2364-7.) These findings along with the lack of availability of sterile hoods in operating rooms underscore the need for a novel closed system for processing, mixing, and reimplanting cells through a percutaneous route.

### SUMMARY OF INVENTION

A system and method is provided for the percutaneous, autologous transplantation of mesenchymal stem cells (MSC's) and progenitor helper cells (PHC) from bone marrow to degenerated intervertebral discs or joints. The systems and methods of the invention are designed to be used by operating room staff in a clinical setting to isolate a MSC population and PHC population during the same surgical procedure as transplantation. The systems and methods can be used as a procedure where target cells are harvested, then isolated, then reimplanted into a target site, all from and into the same patient. The systems and methods further include the use of a novel hyaluronic acid and fibrinogen composite delivered percutaneously through a needle to provide an optimal cell scaffold for the isolated and reimplanted target cells

In addition, experimental techniques are provided to determine which bone marrow cells should be removed via negative selection to generate a MSC/PHC population most likely to regenerate certain tissue types in-vitro as well as which combination of fibrinogen and hyaluronic acid and which degree of gel maceration provides the best matrix for in-vitro and in-vivo regeneration of joints and intervertebral discs.

The present invention also includes a kit based upon a selection device prepared in accordance with the methods described herein. The kit can be used by the operating room personnel to implement the methods of the present invention. Specialized laboratory training is not necessary to use the kit as disclosed herein.

### BRIEF DESCRIPTION OF THE DRAWINGS

FIG. 1 is a schematic diagram of a mixing and maceration chamber consistent with the present invention.

### DETAILED DESCRIPTION OF INVENTION

In one embodiment of the present invention, a closed system is provided to obtain bone marrow samples from a patient while reducing the chance of inadvertent cell contamination and resultant infection. In one embodiment of this system, the physician uses a prepackaged sterile Trocar (or other like device) to draw marrow blood and cells into a sterile prepackaged syringe or container. The operating room (OR) staff or other like user attaches the marrow collection syringe or container to a connector with a large bore needle. The OR staff then inserts the needle into a rubber stopper at the top of prepackaged sterile centrifuge tubes which have been packaged with a vacuum and have a port or opening built into the bottom of the tube. A marrow sample of 50 cc to 400 cc is transferred to the centrifuge tube(s) and the tubes are placed in a medical grade centrifuge (marrow is typically harvested from the iliac crest). The plasma is separated from the cells using the centrifuge and the port at the bottom of each tube is attached to connector tubing and the cells drawn into a syringe through negative pressure created by the plunger of the syringe.

Once all cells have been drawn into the cell collector syringe, this is connected to an isolation column or device containing antibodies to the specific cells (surface markers) which need to be removed by negative selection (the types of cells to be removed to produce the best in-vitro and in-vivo result to be determined by experiment as described herein). The cells are pushed through the isolation column by action of the plunger of the syringe or by any other technique which allows the cells to flow through the column. The isolation column is attached to another syringe (isolated cell syringe) which is filled with the cells not attached in the isolation column. The column is washed with phosphate buffered normal saline which further fills the isolated cell syringe.

In typical embodiments the negative selection column will include antibodies or other like removal agents against CD31 and CD14. Removal of cells having these antigens will remove or reduce the numbers of endothelial cells and monocytes, leaving macrophages, lymphocytes, leudocytes, CD34+ heme progenitors (together referred to as PHC's) and MSC's. In an alternative negative selection column, antibodies or other like agents against CD14, CD11a, CD45, glycophorin A, CD3, CD19, CD34, CD 38 and CD66b provides a cell population of MSC's and CD31+ platelets. Columns themselves can be prepared using beads, microspheres, microbeads, alginate gels and/or magnetic separation technologies.

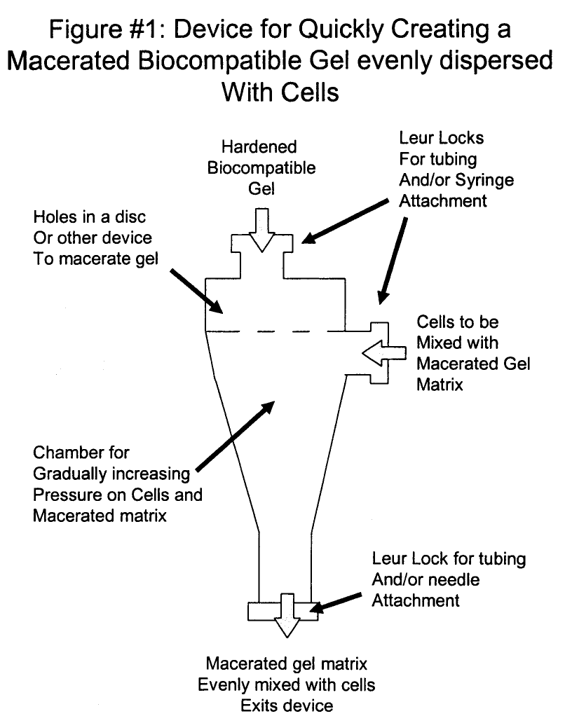

The gel matrix for reimplanation is then mixed in separate syringes by depressing the stopper of a dual syringe containing hyaluronic acid in one syringe and fibrinogen in the other syringe. In some embodiments the hyaluronic acid represents 40-80% of the mix and the fibrinogen represents 60-20% of the mix (by weight), i.e., an embodiment, therefore, can include 40% hyaluronic acid in a one syringe and 60% fibrinogen in the other syringe. This is attached to a matrix collection syringe which is of a special type having a screw device that can be turned to push hardened gel through a needle. As shown in FIG. 1, the bottom of the matrix collection syringe is attached to a specially designed chamber to macerate the gel, mix in cells from the cell collection syringe, and compress the mixture into the delivery needle. The isolated cell syringe is then attached to the side of the specially designed chamber to draw off isolated cells into the macerated gel matrix. The surgeon or other user then turns the screw device of the matrix collection syringe which pushes hardened gel against the macerating plate in the chamber and subsequently mixes cells with the macerated gel. The cells are placed into the mix after the gel has been macerated to protect the cells from trauma. This mixing also ensures an even distribution of cells within the matrix which are then subsequently injected.

Various known cell surface antigens may be targeted for the selection or negative selection of MSCs and PHCs consistent with the techniques described above. Other suitable cell surface antigens may be discovered in the future. A representative list of cell surface antigens which might be suitable for the implementation of the present invention include but are not limited to the cell surface antigens on the following list as well as those listed in Example 1:

### Lymphocytes: CD4, CD 25, CD31, CD38, CD45, CD100, Cd138, CD10, CD8, CD20,

### CD109, CD7, CD19 [1-8]

### Eryothrocytes: CD71 [7]

Monocytes: CD14, CD64, CD13, CD33, c-kit, CD13, CD43 [9] [8, 10-12]

### Basophils/Ganulocytes/Leukocytes: CD217, CD64, CD33, CD13, CD15, 97A6, CD24,

Cd16b, CD35, c-kit, CD11a, CD11b, CD11c, CD25, CD38, CD33, EPO [10-15]

### Macrophages: Cd68, CD11b [16, 17]

### Mast Cells: CD117, 97A6, CD13 [11-13]

### Mononuclear Phagocytes: CD33, CD13, CD15 [10]

### Megakarocytes: CD109 [1]

### Platelets: CD109 [1]

Eosinophils: c-kit [12]

### Stromal Precusors: STRO-1 [18]

Identification and use of the above surface antigens are at least partly based on the following references, each of which is incorporated by reference:

1. Murray, L. J., et al., CD109 is expressed on a subpopulation of CD34+ cells enriched in hematopoietic stem and progenitor cells. Exp Hematol, 1999. 27(8): p. 1282-94.
2. Baecher-Allan, C., E. Wolf, and D. A. Hafler, Functional analysis of highly defined, FACS-isolated populations of human regulatory CD4+ CD25+ T cells. Clin Immunol, 2005. 115(1): p. 10-8.
3. Billard, C., et al., Switch in the protein tyrosine phosphatase associated with human CD100 semaphorin at terminal B-cell differentiation stage. Blood, 2000. 95(3): p. 965-72.
4. Caligiuri, M. A., et al., Functional consequences of interleukin 2 receptor expression on resting human lymphocytes. Identification of a novel natural killer cell subset with high affinity receptors. J Exp Med, 1990, 171(5): p. 1509-26.
5. Gazitt, Y., et al., Purified CD34+Lin−Thy+ stem cells do not contain clonal myeloma cells. Blood, 1995. 86(1): p. 381-9.
6. Medina, F., C. Segundo, and J. A. Brieva, Purification of human tonsil plasma cells: pre-enrichment step by immunomagnetic selection of CD31(+) cells. Cytometry, 2000. 39(3): p. 231-4.
7. Terstappen, L. W., et al., Sequential generations of hematopoietic colonies derived from single nonlineage-committed CD34+CD38-progenitor cells. Blood, 1991. 77(6): p. 1218-27.
8. Otawa, M., et al., Comparative multi-color flow cytometric analysis of cell surface antigens in bone marrow hematopoietic progenitors between refractory anemia and aplastic anemia. Leuk Res, 2000. 24(4): p. 359-66.
9. Ahuja, V., S. E. Miller, and D. N. Howell, Identification of two subpopulations of rat monocytes expressing disparate molecularforms and quantities of CD43. Cell Immunol, 1995. 163(1): p. 59-69.
10. Olweus, J., F. Lund-Johansen, and L. W. Terstappen, CD64/Fc gamma RI is a granulo-monocytic lineage marker on CD34+ hematopoietic progenitor cells. Blood, 1995. 85(9): p. 2402-13.
11. Willheim, M., et al., Purification of human basophils and mast cells by multistep separation technique and mAb to CDw17 and CD17/c-kit. J Immunol Methods, 1995. 182(1): p. 115-29.
12. Kirshenbaum, A. S., et al., Demonstration that human mast cells arise from a progenitor cell population that is CD34(+), c-kit(+), and expresses aminopeptidase N (CD13). Blood, 1999. 94(7): p. 2333-42.
13. Buhring, H. J., et al., The monoclonal antibody 97A6 defines a novel surface antigen expressed on human basophils and their multipotent and unipotent progenitors. Blood, 1999. 94(7): p. 2343-56.
14. Elghetany, M. T. and J. Patel, Assessment of CD24 expression on bone marrow neutrophilic granulocytes: CD24 is a marker for the myelocytic stage of development. Am J Hematol, 2002. 71(4): p. 348-9.
15. Toba, K., et al., Novel technique for the direct flow cytofluorometric analysis of human basophils in unseparated blood and bone marrow, and the characterization of phenotype and peroxidase of human basophils. Cytometry, 1999. 35(3): p. 249-59.
16. Ordog, T., et al., Purification of interstitial cells of Cajal by fluorescence-activated cell sorting. Am J Physiol Cell Physiol, 2004. 286(2): p. C448-56.
17. Hickstein, D. D., et al., Identification of the promoter of the myelomonocytic leukocyte integrin CD11b. Proc Natl Acad Sci U S A, 1992. 89(6): p. 2105-9.
18. Simmons, P. J. and B. Torok-Storb, CD34 expression by stromal precursors in normal human adult bone marrow. Blood, 1991. 78(11): p. 2848-53.

In an alternative embodiment of the present invention, the cell sample may be separated using the same combination of cell surface antigens determined through experimental design discussed herein, with flouresence activated cell sorting being utilized. This alternative selection method may be performed at an on or off-site clinical lab.

Alternatively, the cells selected as most likely to regenerate the target tissue may be expanded in a hospital lab before re-injection.

Alternatively, an open system may be used to collect the marrow and transfer the marrow into centrifuge tubes. The plasma supernatant may be removed once the tubes have been exposed to medical grade centrifugation. The spun cells may be transferred into a separate syringe or container, followed by transfer of these cells to a column or device that isolates mesenchymal stem cells by negative selection as described above. The isolated cells may be washed with PBS normal saline and collected into a syringe which is attached to another syringe containing a mixed hyaluronic acid and fibringogen gel and the two components (isolated MSCs and HA/Fibrinogen gel) pushed through a needle for reimplantation into a degenerated joint or Intervertebral disc. A chamber for macerating the gel, mixing the cells, and preparing cells for injection as described above may be utilized. In this embodiment, a low dose antibiotic may be added to reduce the risk of bacterial contamination or a laminar air-flow surgical suite with appropriate surgical attire may be required if antibiotics are not ultilized.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

Having generally described the invention, the same will be more readily understood by reference to the following examples, which are provided by way of illustration and are not intended as limiting.

### EXAMPLES

### Example 1

### Experiment to Determine which Cells to Negatively Select Out of an Autologous Bone Marrow Sample to Produce the Optimum Combination of MSC's and HPC's to Regenerate Human Intervertebtral Disc or Joints

1. A 50 cc bone marrow sample will be obtained from a donor patient

2. The remaining nucleated cell sample will be processed by a fluorescence activated cell sorter (FACS) to isolate the following populations:
a. Cell Sort #1-Control-no processing
b. Cell Sort #2-Remove Glycophorin A only (exclude RBC's only)
c. Cell Sort#3-Remove 90% of Glycophorin A reactive cells
d. Cell Sort #4-Remove CD31, CD14, CD11a, CD45, glycophorin A (exclude RBC's, endothelial cells, monocytes, macrophages, lymphocytes, leukocytes leaving MSC's and CD 34+ heme progenitors)
e. Cell Sort #5-Remove CD31, CD14, CD11a, CD45, glycophorin A, CD 3, CD 14, CD19, CD34, CD 38, CD66b (exclude all non-MSC's)
f. Cell sort #6-Remove CD14, CD11a, CD45, glycophorin A, CD 3, CD 14, CD19, CD34, CD 38, CD66b (exclude all non MSC's, but leave CD31+ platelets)
g. Cell sort #7-Remove CD31, CD14, CD11a, CD45, glycophorin A, CD 3, CD 14, CD19, CD34, CD 38, CD66b (exclude all non-MSC's) and suspend cells in autologous platelet lystae (created through the collection of Platelet Rich Plasma).

3. Only ½ cc of the above 7 cell populations will be plated with autologous donor H-NPC' s and hyaluronic acid as a carrier agent in hypoxic conditions and under intermittent load (to simulate the absolute amount of injectate that would be practical to inject into a degenerated human intervertebral disc)

4. CFU and assays will be determined for each group at 4 weeks 5. The combination of cell surface markers (using negative selection as described above) that produces the most robust H-NPC colonies will be used in the column or device described above.

6. The above sorting techniques may also be utilized in-vivo to determine which of the in-vitro cell sets produce the best results in-vivo. For example, the three cell sets that produce the best results in-vitro will then be tested by injection into a human IVD or joint.

7. The above experiment (with modifications) will be repeated with human cartilage.

### Example 2

### Experiment to Determine the Appropriate Concentrations and Molecular Weight of Hyaluronic Acid and Fibrinogen and the degree of the Resultant Gel Maceration Needed to Produce the Greatest Number of Colony Forming Units of Human Mesenchymal Stem Cells In-Vitro and In-Vivo

8. A 50 cc bone marrow sample will be obtained from a donor patient

9. The mix of MSC's and HPC's that provide the best in-vitro result above will be used with isolation techniques already described

10. The remaining nucleated cell sample will be processed by a fluorescence activated cell sorter (FACS) to isolate the population of cells that have been determined by the above experiment to produce the best clinical results.

Composite mixtures will consist of:
1. HA: (Hyaluronic Acid 0.5 to 3,000 Kda-1-10 mg/ml
2. Fibrinogen: 5-20 mg/mL

11. These will be seeded into four groups of 1 ml each at a density of 100,000 MSC's combined with autologous nucleus pulposis and chondrocytes (separate experiments):
a. Scaffold #1—Mixture of HA 20%, Fibrinogen 80% 10 mg/ml by volume
b. Scaffold #2—Mixture of HA 40%, Fibrinogen 60% 10 mg/ml by volume
c. Scaffold #3—Mixture of HA 60%, Fibrinogen 40% 10 mg/ml by volume
d. Scaffold #4—Mixture of HA 80%, Fibrinogen 20% 10 mg/ml by volume

12. The cells will be plated at a density of 1.5×10⁵ cells/cm² and placed at 37° C. in a 5% CO₂ incubator. The cells will be exposed to cyclic mechanical loading in a closed system. The culture medium will be changed every other day.

13. CFU and assays will be determined for each group at 4 weeks

14. The above experimental design will then be repeated with hylan A (average molecular weight 6,000,000) and hylan B hydrated gel in a buffered physiological sodium chloride solution, pH 7.2.

15. The HA molecular weight and mixture with fibrinogen that produces the best result will then be forced through maceration devices of various widths to determine which produce the greatest number of colony forming units of human mesenchymal stem cells in-vitro and in-vivo (human NP cells and chondrocytes).

16. The above scaffold designs may also be utilized in-vivo to determine which of the in-vitro cell scaffolds produce the best results clinically. For example, the two cell scaffolds that produce the best results in-vitro will then be tested by injection into a human IVD or joint.

### Example 3

### Various Matrix Combinations Provide Adequate Scaffolding For Proposed MSC Expansion and Differentiation

Sodium hyaluronate (10 mg/ml) was combined with human fibrinogen (10 mg/ml) at different ratios to test the effect of formulation on the viscosity of potential scaffolds for cell injection. The stock solution of fibrinogen was 55-85 mg/ml, and was therefore diluted 1:7 with normal saline to obtain a concentration of approximately 10 mg/ml. Four formulations were evaluated: 20% HA/80% fibrinogen, 40% HA/60% fibrinogen, 60% HA/40% fibrinogen, and 80% HA/20% fibrinogen. 20% HA/80% fibrinogen was the least viscous solution; it was a clear suspension that was easy to pipet. 40% HA/60% fibrinogen exhibited increased viscosity over the 20%/80% formulation and was cloudy in appearance. Despite the increased viscosity it was still relatively easy to pipet. 60% HA/40% fibrinogen was more viscous than the first two formulations, with a cloudy appearance. This solution could be pipetted, but with greater difficulty, resulting in some residue remaining in the pipet tip. 80% HA/20% fibrinogen was the most viscous scaffold, but not as cloudy as the 40/60 and 60/40 mixtures. This solution could not be easily pipetted, with a lot of residue remaining in the pipet tip.

Data from the above formulations showed the following: formulations that include HA at concentrations equal to or higher than 60% may not be ideal from a handling perspective as significant cell suspension may be lost to viscous adhesion to the tissue culture tips and tubes. This could be avoided, however, if the cells are mixed with the scaffold in the injection syringe. Another method that could be utilized to reduce cell loss is to inject the HA/fibrinogen scaffold prior to the cell injection, and then combine the cells with a less viscous HA/fibrinogen solution.

Certain formulations appeared cloudy, suggesting the presence of precipitates. Interestingly, these precipitates were most prominent when HA and fibrinogen were mixed in near equal proportions. It is unclear whether the precipitate is HA or fibrinogen, but suspect it is fibrinogen.

It will be clear that the invention is well adapted to attain the ends and advantages mentioned as well as those inherent therein. While a presently preferred embodiment has been described for purposes of this disclosure, various changes and modifications may be made which are well within the scope of the invention. Numerous other changes may be made which will readily suggest themselves to those skilled in the art and which are encompassed in the spirit of the invention disclosed herein and as defined in the appended claims.

What is claimed is:

1. A method for facilitating cartilage repair in a patient in need thereof comprising:
removing bone marrow containing mesenchymal stem cells (MSC's) from the patient;
negatively selecting for MSCs in the bone marrow wherein a portion of the MSC's in the bone marrow are removed and concentrated; and
re-implanting the concentrated MSC's into a site in the patient in need thereof.

2. The method of claim 1 wherein the removal, negative selection and re-implantation steps are performed in conjunction with the same surgical procedure.

3. The method of claim 1 further comprising negatively selecting other autologous stromal cells.

4. The method of claim 3, wherein the selected autologous stromal cells are progenitor helper cells (PHCs).

5. The method of claim 4 wherein the selected PHCs are selected to support the in-vitro and in-vivo growth of MSCs for the purposes of regenerating at least one of degenerated interverbetral discs, spinal joints, or peripheral joints.

6. The method of claim 1 wherein the MSCs are selected for the purposes of regenerating at least one of degenerated interverbetral discs, spinal joints, or peripheral joints.

7. The method of claim 1, wherein the selection step is performed by an operating surgeon or an operating room staff member.

8. The method of claim 1, wherein the selection step comprises isolation of specific populations of human stromal cells using negative selection.

9. The method of claim 1, whereby the bone marrow is removed by the operating surgeon using a Trocar.

10. The method of claim 1, further comprising separating plasma withdrawn in conjunction with the bone marrow from the bone marrow.

11. The method of claim 10 wherin the plasmas separation step comprises:
placing the bone marrow into at least one medical grade centrifuge tube and
spinning the bone marrow in a centrifuge to separate MSCs and PHCs from plasma.

12. The method of claim 1 wherein the negative selection step is performed with a selection device having antibodies against CD31 and CD14.

13. The method of claim 12 wherein the selection device contains at least one of beads, microspheres, flasks, magnetic particles, immunorosettes, and similar substrates suitable for supporting the process of immunoadsorption to bind non-MSC's or non-PHC's with the cell surface antigens CD31 and CD14.

14. The method of claim 1 wherein the negative selection step is performed with a selection device having antibodies selected from a group including one of or a combination of CD31, CD14, CD11a, CD45, glycophorin A, CD3, CD14, CD19, CD34, CD38 and CD66b.

15. The method of claim 14 wherein the cell surface antigen or the combination of cell surface antigens is chosen by in-vitro experiment to provide the best result in regenerating human intervertebral discs or cartilage.

16. The method of claim 12 further comprising rinsing the selection device with at least one of a phosphate buffered saline or other inert rinsing agent and a reagent to deactivate binding of antibodies and antigens.

17. The method of claim 1 further comprising collecting cells which pass through the negative selection step in a container for reimplantation.

18. The method of claim 17, further comprising mixing the collected cells with a matrix carrier for reimplantation.

19. The method of claim 18 wherein the collected cells are mixed with fibrinogen and hyaluronic acid to produce a gel compound.

20. The method of claim 19 wherein the fibrinogen is from about 20-60%, by weight, of the matrix carrier and the hyaluronic acid is from about 40-80% of the carrier, by weight.

21. The method of claim 1 wherein the site of the patient in need thereof is at least one of a intervertebral disc space and a joint.

22. The method of claim 21 further comprising
sealing the re-implantation site in the patient with a fibrin glue to retain injected cells in the disc or joint.

23. The method of claim 22, wherein the intervertebral disc space or joint is accessed through use of a large bore introducer needle and then a smaller disc entry needle placed within the introducer.

24.-30. (canceled)
